(19) United States
(12) Patent Application Publication
(10) Pub. No.: US 2007/0016008 A1
(43) Pub. Date: Jan. 18, 2007

(54) Selective gesturing input to a surgical navigation system

(76) Inventor: Ryan Schoenefeld, Fort Wayne, IN (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/290,267

(22) Filed: Nov. 30, 2005

Related U.S. Application Data
(60) Provisional application No. 60/693,461, filed on Jun. 23, 2005.

(51) Int. Cl. A61B 5/05

(52) U.S. Cl. 600/424

(57) ABSTRACT

A surgical navigation system uses selective gesturing within a sterile field to provide inputs to a computer, which can reduce surgery time and costs. The teachings comprise configuring an array with at least a first marker and a second marker; exposing the array to a measurement field of the tracking system; occluding the exposure of either the first marker or the second marker to the tracking system within the sterile field; and assigning the occlusion of the first marker as a first input and assigning the occlusion of the second marker as a second input to the computer system, wherein the first input is different than the second input.

### RELATED APPLICATIONS

This application claims priority to U.S. provisional application Ser. No. 60/693,461, filed Jun. 23, 2005.

### FIELD OF THE INVENTION

The present teachings relate to surgical navigation and more particularly to clinicians inputting information into a surgical navigation system.

### BACKGROUND

Surgical navigation systems, also known as computer assisted surgery and image guided surgery, aid surgeons in locating patient anatomical structures, guiding surgical instruments, and implanting medical devices with a high degree of accuracy. Surgical navigation has been compared to a global positioning system that aids vehicle operators to navigate the earth. A surgical navigation system typically includes a computer, a tracking system, and patient anatomical information. The patient anatomical information can be obtained by using an imaging mode such a fluoroscopy, computer tomography (CT) or by simply defining the location of patient anatomy with the surgical navigation system. Surgical navigation systems can be used for a wide variety of surgeries to improve patient outcomes.

Surgical navigation systems can receive inputs to operate a computer from a keypad, touch screen, and gesturing. Gesturing is where a surgeon or clinician manipulates or blocks a tracking system's recognition of an array marker, such as an instrument array marker, to create an input that is interpreted by a computer system. For example, a clinician could gesture by temporarily occluding one or more of the markers on an array from a camera for a period of time so that the temporary occlusion is interpreted by the computer as an input. The computer system could recognize the gesture with a visual or audio indicator to provide feedback to the clinician that the gesture has been recognized. The computer system's interpretation of the gesture can depend upon the state of the computer system or the current operation of the application program. Current gesturing techniques create a single input from an array for the computer. It would be desirable to improve upon these gesturing techniques to reduce surgery time and costs.

### SUMMARY OF THE INVENTION

Selective gesturing input to a surgical navigation system within a sterile field can reduce surgery time and costs. The teachings comprise configuring an array with a first marker and a second marker, wherein the first marker and second marker are distinguishable by a tracking system; exposing the array to a measurement field of the tracking system; occluding the exposure of either the first marker or the second marker to the tracking system within the sterile field; assigning the occlusion of the first marker as a first input and assigning the occlusion of the second marker as a second input to the computer system, wherein the first input is different than the second input. The teachings can have a wide range of embodiments including embodiments on a computer readable storage medium.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, wherein:

FIG. 1 is a perspective view of an operating room setup in a surgical navigation embodiment in accordance with the present teachings;

FIG. 2 is a block diagram of a surgical navigation system embodiment in accordance with the present teachings;

FIGS. 2A-2G are block diagrams further illustrating the surgical navigation system embodiment of FIG. 2;

FIG. 3 is a first exemplary computer display layout embodiment in accordance with the present teachings;

FIG. 4 is a second exemplary computer display layout embodiment;

FIG. 5 is an exemplary surgical navigation kit embodiment in accordance with the present teachings;

FIGS. 6A and 6B are perspective views of an exemplary calibrator array embodiment in accordance with the present teachings;

FIG. 7 is a flowchart illustrating the operation of an exemplary surgical navigation system in accordance with the present teachings;

FIGS. 8-10 are flowcharts illustrating exemplary selective gesturing embodiments in accordance with the present teachings; and

FIGS. 11A-11D are fragmentary perspective views illustrating an example of an exemplary method in accordance with the present teachings.

Corresponding reference characters indicate corresponding parts throughout the several views.

### DETAILED DESCRIPTION

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

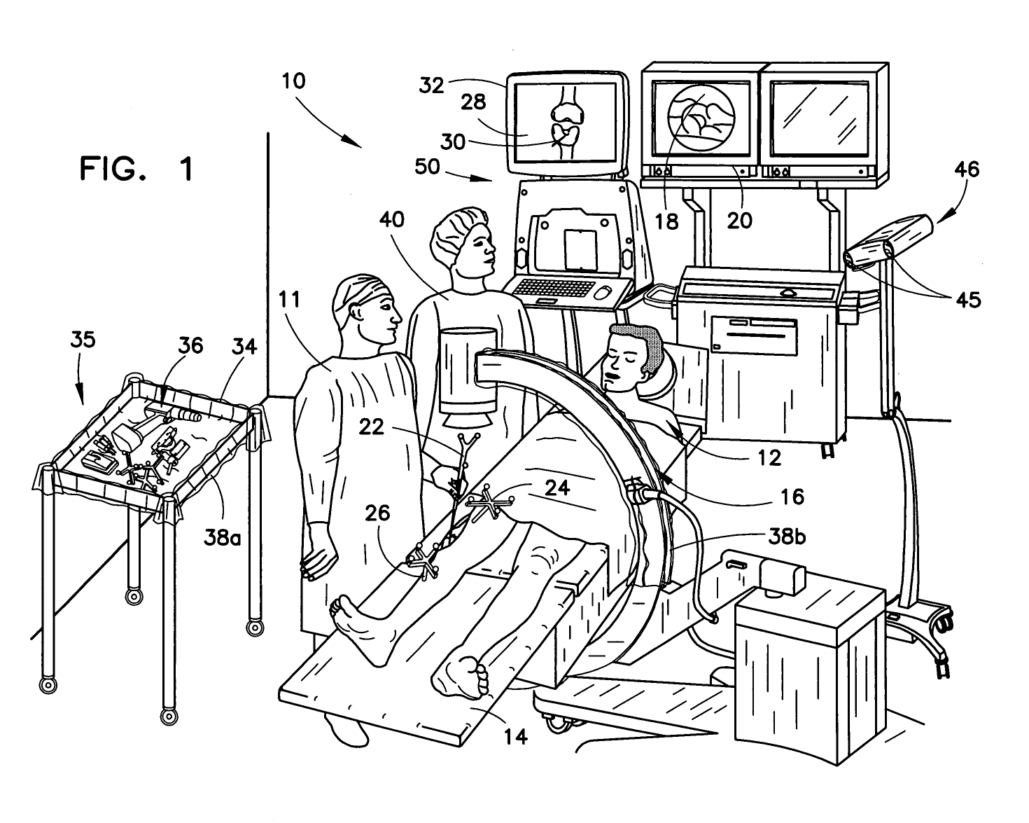

FIG. 1 shows a perspective view of an operating room with a surgical navigation system 10. Surgeon 11 is aided by the surgical navigation system in performing knee arthroplasty, also known as knee replacement surgery, on patient 12 shown lying on operating table 14. Surgical navigation system 10 has a tracking system that locates arrays and tracks them in real time. To accomplish this, the surgical navigation system includes optical locator 46, which has two CCD (charge couple device) cameras 45 that detect the positions of the arrays in space by using triangulation methods. The relative location of the tracked arrays, including the patient's anatomy, can then be shown on a computer display (such as computer display 50 for instance) to assist the surgeon during the surgical procedure. The arrays that are typically used include probe arrays, instrument arrays, reference arrays, and calibrator arrays. The operating room includes an imaging system such as C-arm fluoroscope 16 with fluoroscope display image 18 to show a real-time image of the patient's knee on monitor 20. Surgeon 11 uses surgical probe 22 to reference a point on the patient's knee, and reference arrays 24, 26 attached to the patient's femur and tibia to provide known anatomic reference points so the surgical navigation system can compensate for leg movement. The relative location of probe array 22 to the patient's tibia is then shown as reference numeral 30 on computer display image 28 of computer monitor 32. The operating room also includes instrument cart 35 having tray 34 for holding a variety of surgical instruments and arrays 36. Instrument cart 35 and C-arm 16 are typically draped in sterile covers 38a, 38b to eliminate contamination risks within the sterile field.

The surgery is performed within a sterile field, adhering to the principles of asepsis by all scrubbed persons in the operating room. Patient 12, surgeon 11 and assisting clinician 40 are prepared for the sterile field through appropriate scrubbing and clothing. The sterile field will typically extend from operating table 14 upward in the operating room. Typically both computer display image 28 and fluoroscope display image 18 are located outside of the sterile field.

A representation of the patient's anatomy can be acquired with an imaging system, a virtual image, a morphed image, or a combination of imaging techniques. The imaging system can be any system capable of producing images that represent the patient's anatomy such as a fluoroscope producing x-ray two-dimensional images, computer tomography (CT) producing a three-dimensional image, magnetic resonance imaging (MRI) producing a three-dimensional image, ultrasound imaging producing a two-dimensional image, and the like. A virtual image of the patient's anatomy can be created by defining anatomical points with the surgical navigation system 10 or by applying a statistical anatomical model. A morphed image of the patient's anatomy can be created by combining an image of the patient's anatomy with a data set, such as a virtual image of the patient's anatomy. Some imaging systems, such as a C-arm fluoroscope 16, can require calibration. The C-arm can be calibrated with a calibration grid that enables determination of fluoroscope projection parameters for different orientations of the C-arm to reduce distortion. A registration phantom can also be used with a C-arm to coordinate images with the surgical navigation application program and improve scaling through the registration of the C-arm with the surgical navigation system. A more detailed description of a C-arm based navigation system is provided in James B. Stiehl et al., Navigation and Robotics in Total Joint and Spine Surgery, Chapter 3 C-Arm-Based Navigation, Springer-Verlag (2004).

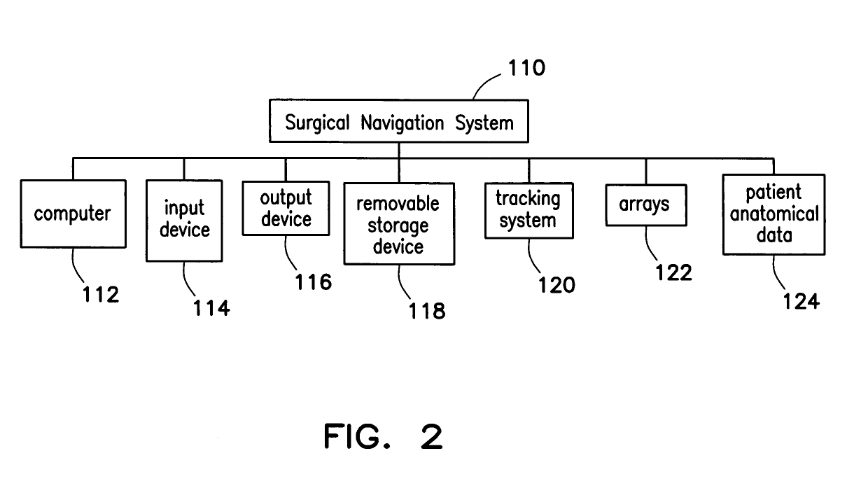

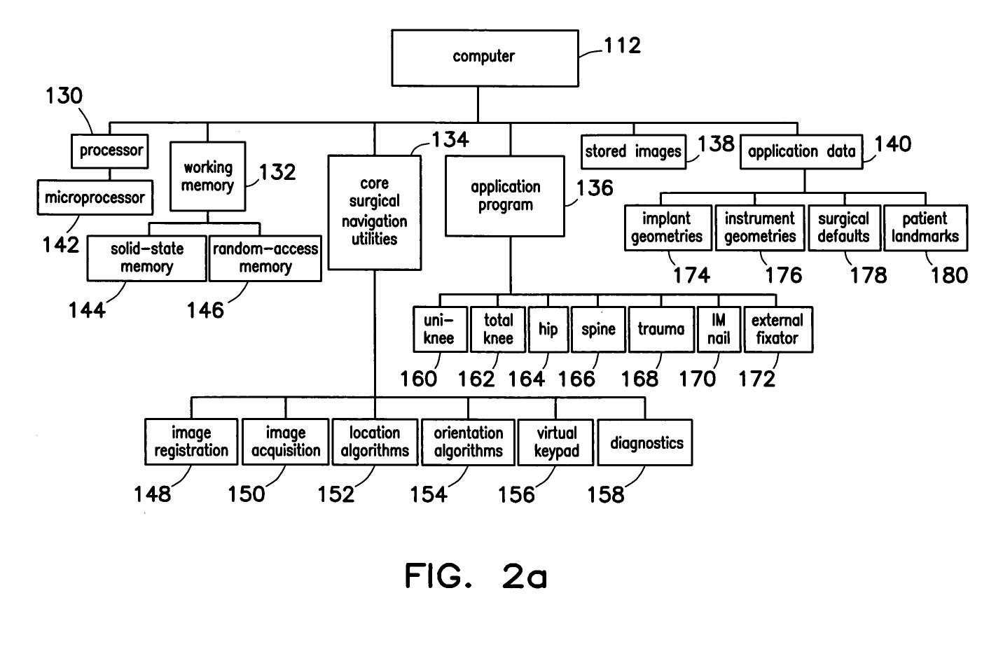

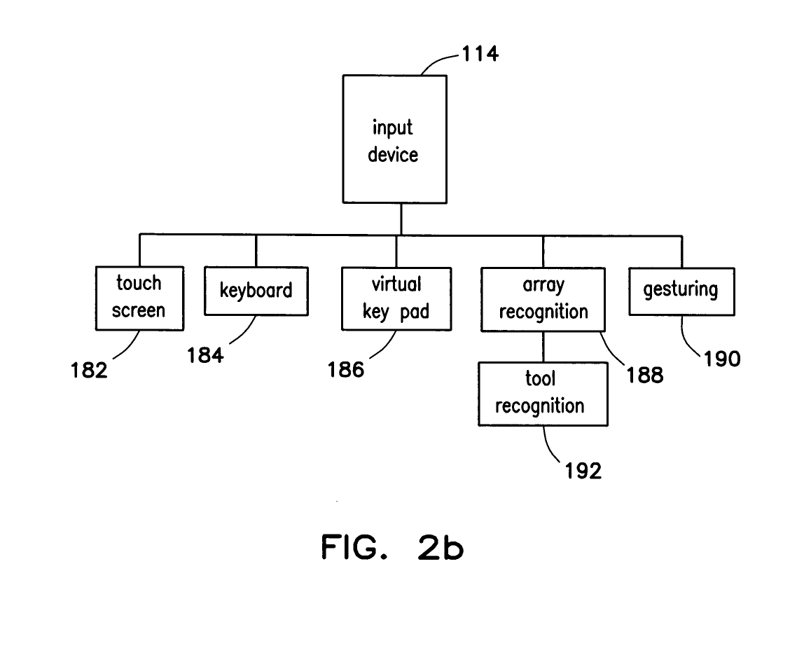

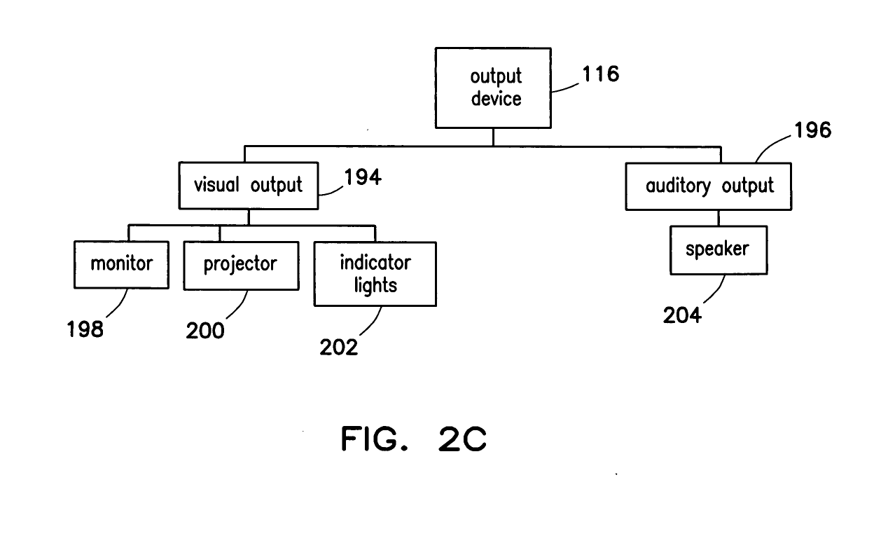

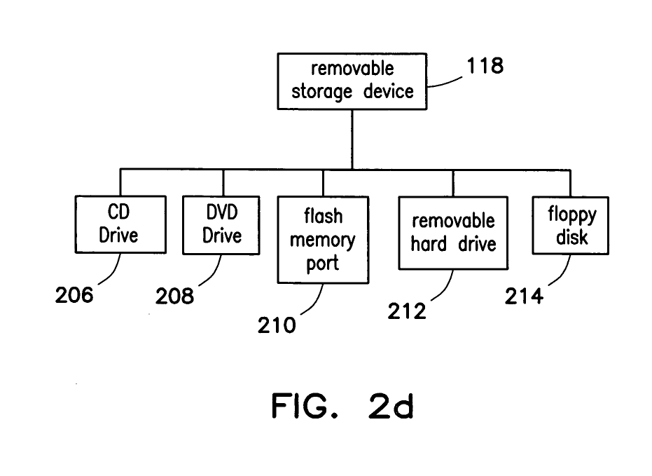

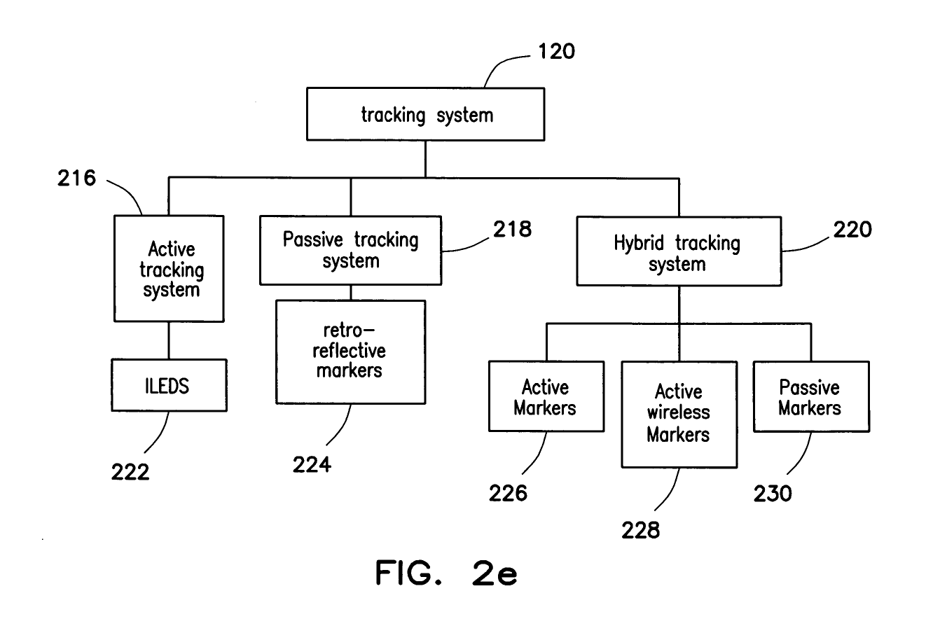

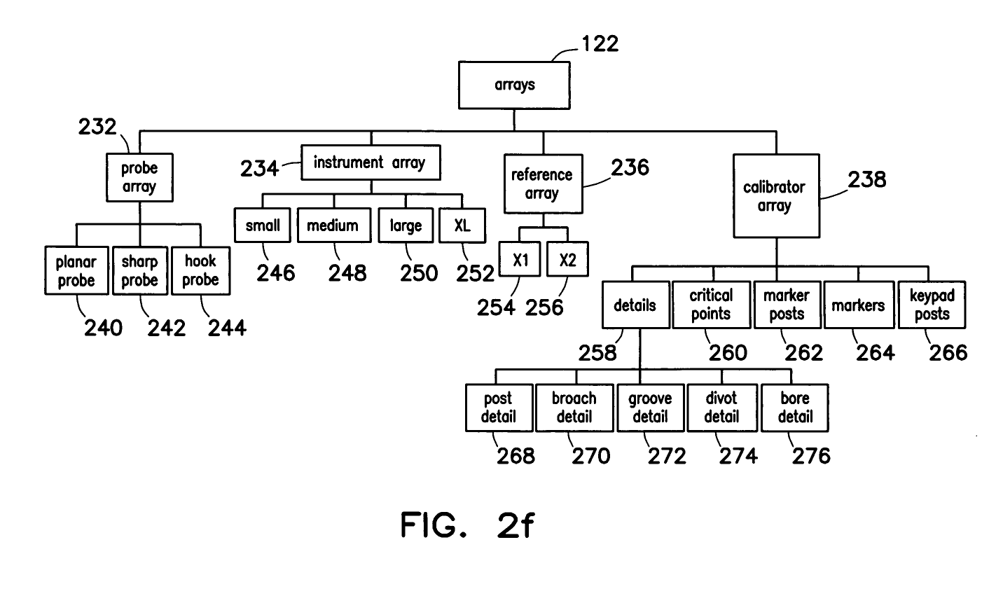

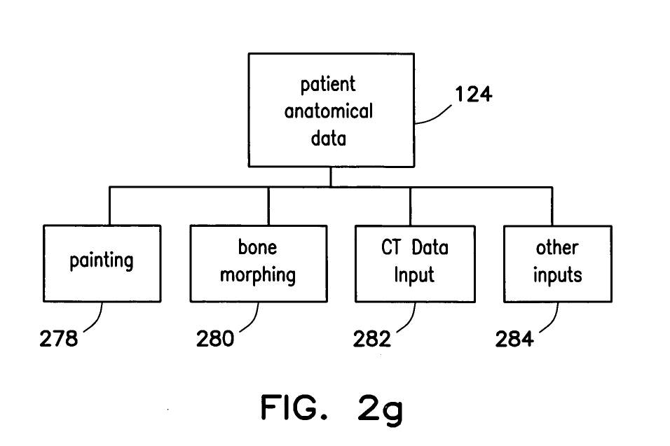

FIG. 2 is a block diagram of an exemplary surgical navigation system embodiment in accordance with the present teachings, such as an Acumen™ Surgical Navigation System available from EBI, L.P., Parsipanny, N.J. USA, a Biomet Company. The surgical navigation system 110 comprises computer 112, input device 114, output device 116, removable storage device 118, tracking system 120, arrays 122, and patient anatomical data 124, as further described in the brochure Acumen™ Surgical Navigation System, Understanding Surgical Navigation (2003), available from EBI, L.P. The Acumen™ Surgical Navigation System can operate in a variety of imaging modes such as a fluoroscopy mode creating a two-dimensional x-ray image, a computer-tomography (CT) mode creating a three-dimensional image, and an imageless mode creating a virtual image or planes and axes by defining anatomical points of the patient's anatomy. In the imageless mode, a separate imaging device such as a C-arm is not required, thereby simplifying set-up. The Acumen™ Surgical Navigation System can run a variety of orthopedic applications, including applications for knee arthroplasty, hip arthroplasty, spine surgery, and trauma surgery, as further described in the brochure “Acumen™ Surgical Navigation System, Surgical Navigation Applications” (2003) available from EBI, L.P. A more detailed description of an exemplary surgical navigation system is provided in James B. Stiehl et al., Navigation and Robotics in Total Joint and Spine Surgery, Chapter 1 Basics of Computer-Assisted Orthopedic Surgery (CAOS), Springer-Verlag (2004).

As depicted in FIG. 2a, computer 112 can be any computer capable of property operating surgical navigation devices and software, such as a computer similar to a commercially available personal computer that comprises processor 130, working memory 132, core surgical navigation utilities 134, an application program 136, stored images 138, and application data 140. Processor 130 is a processor of sufficient power for computer 112 to perform desired functions, such as one or more microprocessors 142. Working memory 132 is memory sufficient for computer 112 to perform desired functions such as solid-state memory 144, random-access memory 146, and the like. Core surgical navigation utilities 134 are the basic operating programs, and include image registration 148, image acquisition 150, location algorithms 152, orientation algorithms 154, virtual keypad 156, diagnostics 158, and the like. Application program 136 can be any program configured for a specific surgical navigation purpose, such as orthopedic application programs for unicondylar knee (“uni-kee”) 160, total knee 162, hip 164, spine 166, trauma 168, intramedullary (“IM”) nail 170, and external fixator 172. Stored images 138 are those recorded during image acquisition using any of the imaging systems previously discussed. Application data 140 is data that is generated or used by application program 136 such as implant geometries 174, instrument geometries 176, surgical defaults 178, patient landmarks 180, and the like. Application data 140 can be pre-loaded in the software or input by the user during a surgical navigation procedure.

As depicted in FIG. 2b, input device 114 can be any device capable of interfacing between a clinician and the computer system such as touch screen 182, keyboard 184, virtual keypad 186, array recognition 188, gesturing 190, and the like. The touch screen typically covers the computer display and has buttons configured for the specific application program 136. Touch screen 182 can be operated by a clinician outside of the sterile field or by a surgeon or clinician in the sterile field with the aid of a sterile drape or sterile stylus. Keyboard 184 is typically closely associated with computer 112 and can be directly attached to computer 112. Virtual keypad 186 is a template having marked areas that correspond to commands for application program 136 that is coupled to an array, such as a calibrator array. Array recognition 188 is a feature where the surgical navigation system 110 recognizes a specific array when the array is exposed to the measurement field. Array recognition 188 allows computer 112 to identify specific arrays and take appropriate actions in application program 136. One specific type of array recognition 188 is recognition of an array attached to an instrument, which is also known as tool recognition 192. When a clinician picks up an instrument with an attached instrument array, the instrument is automatically recognized by the computer system, and application program 136 can automatically advance to the portion of the application where this instrument is used.

As shown in FIG. 2c, output device 116 can be any device capable of creating an output useful for surgery, such as visual output 194 and auditory output 196. Visual output device 194 can be any device capable of creating a visual output useful for surgery, such as a two-dimensional image, a three-dimensional image, a holographic image, and the like. The visual output device can be monitor 198 for producing two and three-dimensional images, projector 200 for producing two and three-dimensional images, and indicator lights 202. Auditory output 196 can be any device capable of creating an auditory output used for surgery, such as speaker 204 that can be used to provide a voice or tone output.

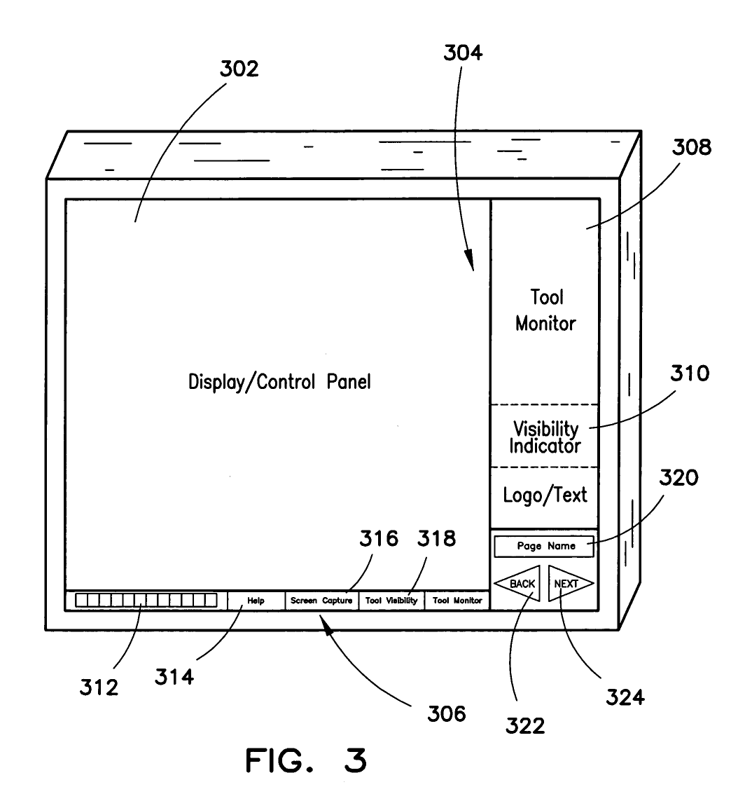

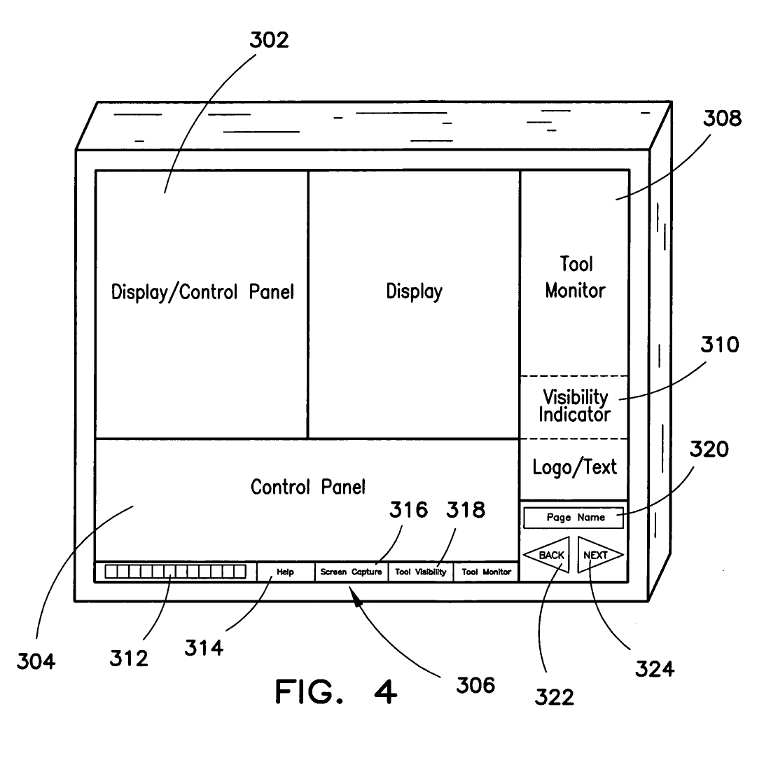

FIG. 3 shows a first computer display layout embodiment, and FIG. 4 shows a second computer display layout embodiment in accordance with the present teachings. The display layouts can be used as a guide to create common display topography for use with various embodiments of input devices 114 and to produce visual outputs 194 for core surgical navigation utilities 134, application programs 136, stored images 138, and application data 140 embodiments. Each application program 136 is typically arranged into sequential pages of surgical protocol that are configured according to a graphic user interface scheme. The graphic user interface can be configured with main display 302, main control panel 304, and tool bar 306. Main display 302 presents images such as selection buttons, image viewers, and the like. Main control panel 304 can be configured to provide information such as tool monitor 308, visibility indicator 310, and the like. Tool bar 306 can be configured with status indicator 312, help button 314, screen capture button 316, tool visibility button 318, current page button 320, back button 322, forward button 324, and the like. Status indicator 312 provides a visual indication that a task has been completed, visual indication that a task must be completed, and the like. Help button 314 initiates a pop-up window containing page instructions. Screen capture button 316 initiates a screen capture of the current page, and tracked elements will display when the screen capture is taken. Tool visibility button 318 initiates a visibility indicator pop-up window or adds a tri-planar tool monitor to control panel 304 above current page button 320. Current page button 320 can display the name of the current page and initiate a jump-to menu when pressed. Forward button 324 advances the application to the next page. Back button 322 returns the application to the previous page. The content in the pop-up will be different for each page.

Referring now to FIG. 2d, removable storage device 118 can be any device having a removable storage media that would allow downloading data such as application data and patient data. The removable storage device can be read-write compact disc (CD) drive 206, read-write digital video disc (DVD) drive 208, flash solid-state memory port 210, removable hard drive 212, floppy disc drive 214, and the like.

As shown in FIG. 2e, tracking system 120 can be any system that can determine the three-dimensional location of devices carrying or incorporating markers that serve as tracking indicia. Active tracking system 216 has a collection of infrared light emitting diodes (ILEDs) 222 illuminators that surround the position sensor lenses to flood a measurement field of view with infrared light. Passive system 218 incorporates retro-reflective markers 224 that reflect infrared light back to the position sensor, and the system triangulates the real-time position (x, y, and z location) and orientation (rotation around x, y, and z axes) of an array and reports the result to the computer system with an accuracy of about 0.35 mm Root Mean Squared (RMS). An example of passive tracking system 218 is a Polaris® Passive System and an example of a marker is the NDI Passive Spheres™ both available from Northern Digital Inc. Ontario, Canada. Hybrid, tracking system 220 can detect active 226 and active wireless markers 228 in addition to passive markers 230. Active marker based instruments enable automatic tool identification, program control of visible LEDs, and input via tool buttons. An example of hybrid tracking system 220 is the Polaris® Hybrid System available from Northern Digital Inc. A marker can be a passive IR reflector, an active IR emitter, an electromagnetic marker, and an optical marker used with an optical camera.

As shown in FIG. 2f, arrays 122 can be probe arrays 232, instrument arrays 234, reference arrays 236, calibrator arrays 238, and the like. Array 122 can have any number of markers, but typically have three or more markers to define real-time position (x, y, and z location) and orientation (rotation around x, y, and z axes). An array comprises a body and markers. The body comprises an area for spatial separation of markers. In some embodiments, there are at least two arms and some embodiments can have three arms, four arms, or more. The arms are typically arranged asymmetrically to facilitate specific array and marker identification by the tracking system. In other embodiments, such as a calibrator array, the body provides sufficient area for spatial separation of markers without the need for arms. Arrays can be disposable or non-disposable. Disposable arrays are typically manufactured from plastic and include installed markers. Non-disposable arrays are manufactured from a material that can be sterilized, such as aluminum, stainless steel, and the like. The markers are removable, so they can be removed before sterilization.

Probe arrays 232 can have many configurations such as planar probe 240, sharp probe 242, and hook probe 244. Sharp probe 242 is used to select patient anatomical discrete points for discrete anatomical landmarks that define points and planes in space for system calculations and surgical defaults. Hook probe 244 is typically used to acquire data points in locations where sharp probe 242 would be awkward such as in unicondylar knee applications. Planar probe 240 is used to define planes such as a cut block plane for tibial resection, varus-valgus planes, tibial slope planes, and the like. Probe arrays 232 have two or more markers arranged asymmetrically, so the tracking system can recognize the specific probe array.

Instrument arrays 234 can be configured in many ways such as small instrument array 246, medium instrument array 248, large instrument array 250, extra-large instrument array 252, and the like. Instrument arrays have array attachment details for rigidly attaching the instrument array to an instrument. Reference arrays 236 can be configured in many ways such as X1 reference array 254, X2 reference array 256, and the like. Reference arrays 236 also have at least one array attachment detail for attaching the reference array to human anatomy with a device, such as a bone anchor or for attaching the reference array to another desired reference such as an operating table, and the like.

Calibrator arrays comprise calibrator details 258, calibrator critical points 260, marker posts 262, markers 264, and keypad posts 266. Calibrator details 258 include a post detail 268, broach detail 270, groove detail 272, divot detail 274, and bore detail 276.

Referring to FIG. 2g, planning and collecting patient anatomical data 124 is a process by which a clinician inputs into the surgical navigation system actual or approximate anatomical data. Anatomical data can be obtained through techniques such as anatomic painting 278, bone morphing 280, CT data input 282, and other inputs 284, such as ultrasound and fluoroscope and other imaging systems.

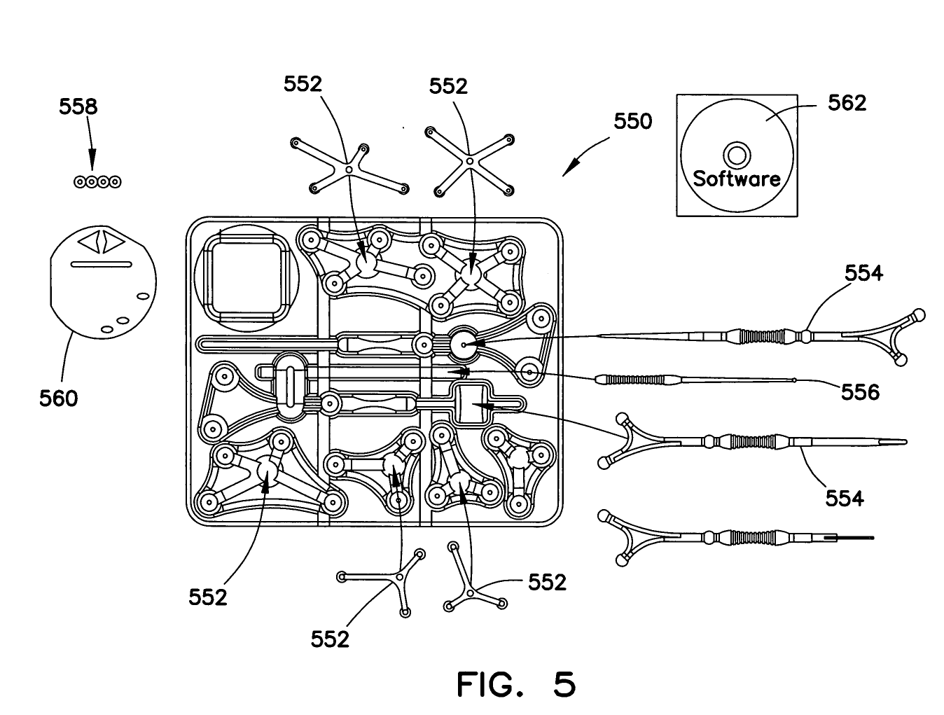

FIG. 5 shows orthopedic application kit 550, which is used in accordance with the present teachings. Application kit 550 is typically carried in a sterile bubble pack and is configured for a specific surgery. Exemplary kit 550 comprises arrays 552, surgical probes 554, stylus 556, markers 558, virtual keypad template 560, and application program 562. Orthopedic application kits are available for unicondylar knee, total knee, total hip, spine, and external fixation from EBI, L.P.

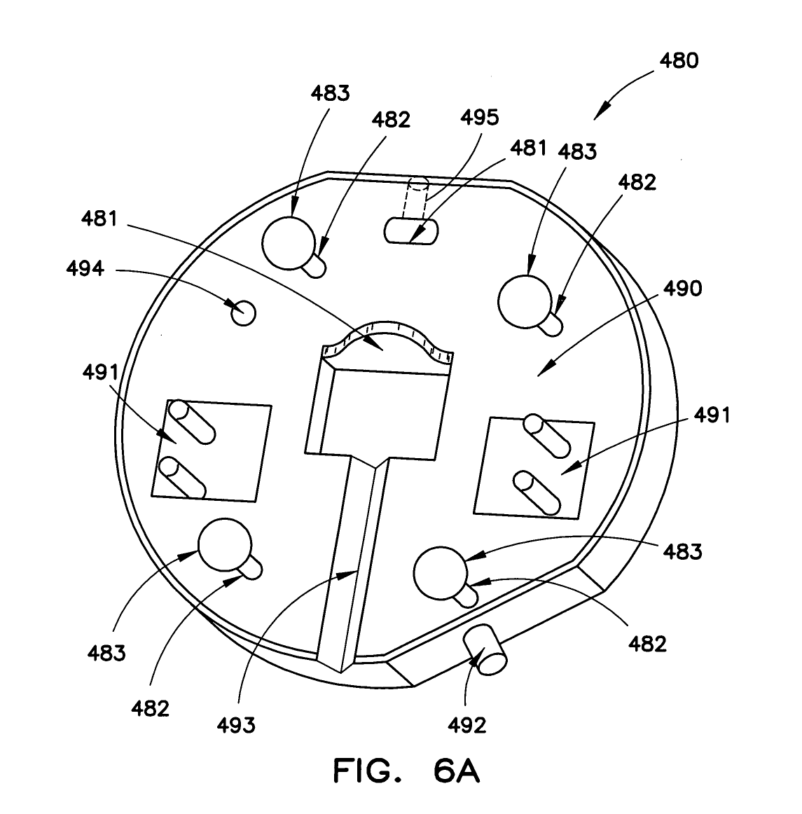

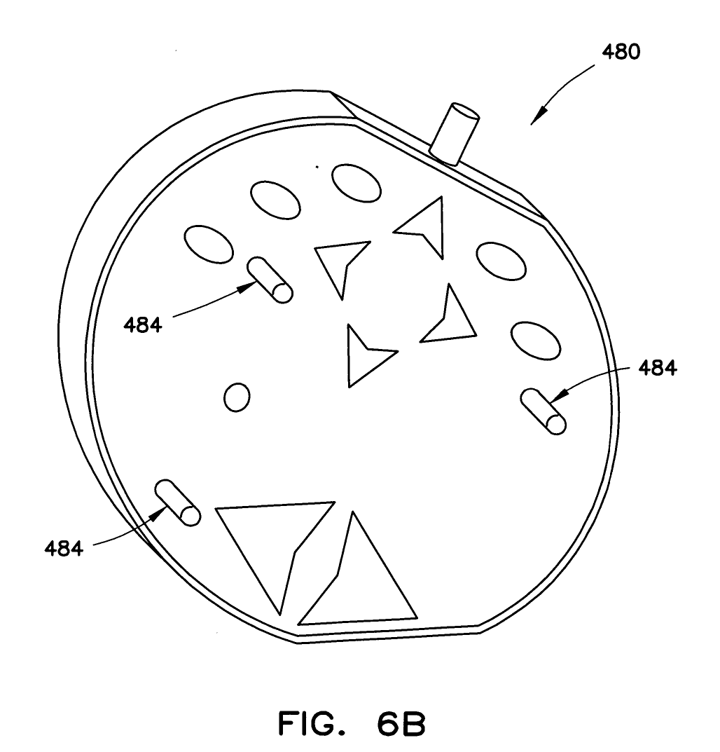

FIGS. 6A and 6B respectively show front and back perspectives of an exemplary calibration array embodiment in accordance with the present teachings. During set-up, instruments having an instrument array attached typically require registration with a calibration array. Calibrator array 480 is a device used to input into the tracking system instrument critical points, so the tracking system can accurately track the instrument. As explained above, calibrator array 480 comprises calibrator details 490, calibrator critical points 481, marker posts 482, markers 483, and keypad posts 484. Calibrator details 490 include post detail 491, broach detail 492, groove detail 493, divot detail 494, and bore detail 495. When an instrument array is attached to an instrument, the system does not know the directional or spatial orientation of the instrument with respect to the instrument array. Calibration defines that orientation. Calibration critical points 490 are programmed into the computer that once mated with an instrument critical point establishes a fiducial relationship among calibrator 480, the instrument, and the application program. The software defines which calibrator critical point 481 corresponds to each instrument that will be tracked by the system. Each calibrator critical point 481 corresponds to a calibration detail 490. Post detail 491 is used for static calibration of instruments and to stabilize other instruments during calibration. Broach detail 492 is used to statically calibrate an instrument such as a broach handle, and the like. Divot detail 494 is used for pivoting calibration of an instrument such as a burr for a unicondular knee application, and the like. Bore detail 495 and groove detail 493 are used to define an instrument axis and critical points such as for an acetabular cup impactor, pedicle screw inserter, and the like. Marker posts 482 receive markers 483 that function as an array to identify calibrator 480 and its location to the tracking system. Markers 483 are removable from marker posts 482, so the calibrator array can be sterilized through a process such as an autoclave without damaging the markers. Keypad posts 484 provide attachment structure for a virtual key pad.

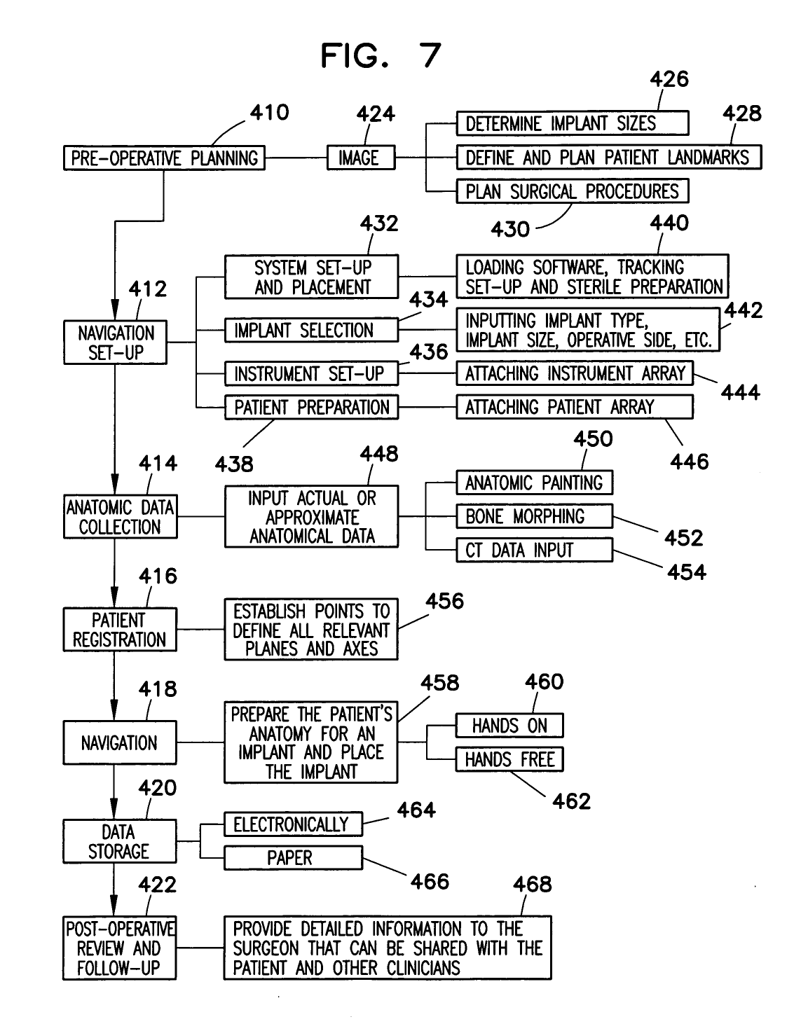

FIG. 7 shows an operational flowchart of a surgical navigation system in accordance with the present teachings. The process of surgical navigation can include the elements of pre-operative planning 410, navigation set-up 412, anatomic data collection 414, patient registration 416, navigation 418, data storage 420, and post-operative review and follow-up 422.

Pre-operative planning 410 is performed by generating an image 424, such as a CT scan that is imported into the computer. With image 424 of the patient's anatomy, the surgeon can then determine implant sizes 426, such as screw lengths, define and plan patient landmarks 428, such as long leg mechanical axis, and plan surgical procedures 430, such as bone resections and the like. Pre-operative planning 410 can reduce the length of intra-operative planning thus reducing overall operating room time.

Navigation set-up 412 includes the tasks of system set-up and placement 432, implant selection 434, instrument set-up 436, and patient preparation 438. System set-up and placement 432 includes loading software, tracking set-up, and sterile preparation 440. Software can be loaded from a pre-installed application residing in memory, a single use software disk, or from a remote location using connectivity such as the internet. A single use software disk contains an application that will be used for a specific patient and procedure that can be configured to time-out and become inoperative after a period of time to reduce the risk that the single use software will be used for someone other than the intended patient. The single use software disk can store information that is specific to a patient and procedure that can be reviewed at a later time. Tracking set-up involves connecting all cords and placement of the computer, camera, and imaging device in the operating room. Sterile preparation involves placing sterile plastic on selected parts of the surgical navigation system and imaging equipment just before the equipment is moved into a sterile environment, so the equipment can be used in the sterile field without contaminating the sterile field.

Navigation set-up 412 is completed with implant selection 434, instrument set-up 436, and patient preparation 438. Implant selection 434 involves inputting into the system information such as implant type, implant size, patient size, operative side and the like 442. Instrument set-up 436 involves attaching an instrument array to each instrument intended to be used and then calibrating each instrument 444. Instrument arrays should be placed on instruments, so the instrument array can be acquired by the tracking system during the procedure. Patient preparation 438 is similar to instrument set-up because an array is typically rigidly attached to the patient's anatomy 446. Reference arrays do not require calibration but should be positioned so the reference array can be acquired by the tracking system during the procedure.

As mentioned above, anatomic data collection 414 involves a clinician inputting into the surgical navigation system actual or approximate anatomical data 448. Anatomical data can be obtained through techniques such as anatomic painting 450, bone morphing 452, CT data input 454, and other inputs, such as ultrasound and fluoroscope and other imaging systems. The navigation system can construct a bone model with the input data. The model can be a three-dimensional model or two-dimensional pictures that are coordinated in a three-dimensional space. Anatomical painting 450 allows a surgeon to collect multiple points in different areas of the exposed anatomy. The navigation system can use the set of points to construct an approximate three-dimensional model of the bone. The navigation system can use a CT scan done pre-operatively to construct an actual model of the bone. Fluoroscopy uses two-dimensional images of the actual bone that are coordinated in a three-dimensional space. The coordination allows the navigation system to accurately display the location of an instrument that is being tracked in two separate views. Image coordination is accomplished through a registration phantom that is placed on the image intensifier of the C-arm during the acquisition of images. The registration phantom is a tracked device that contains imbedded radio-opaque spheres. The spheres have varying diameters and reside on two separate planes. When an image is taken, the fluoroscope transfers the image to the navigation system. Included in each image are the imbedded spheres. Based on previous calibration, the navigation system is able to coordinate related anterior and posterior views and coordinate related medial and lateral views. The navigation system can also compensate for scaling differences in the images.

Patient registration 416 establishes points that are used by the navigation system to define all relevant planes and axes 456. Patient registration 416 can be performed by using a probe array to acquire points, placing a software marker on a stored image, or automatically by software identifying anatomical structures on an image or cloud of points. Once registration is complete, the surgeon can identify the position of tracked instruments relative to tracked bones during the surgery. The navigation system enables a surgeon to interactively reposition tracked instruments to match planned positions and trajectories and assists the surgeon in navigating the patient's anatomy.

During the procedure, step-by-step instructions for performing the surgery in the application program are provided by a navigation process. Navigation 418 is the process a surgeon uses in conjunction with a tracked instrument or other tracked array to precisely prepare the patient's anatomy for an implant and to place the implant 458. Navigation 418 can be performed hands-on 460 or hands-free 462. However navigation 418 is performed, there is usually some form of feedback provided to the clinician such as audio feedback or visual feedback or a combination of feedback forms. Positive feedback can be provided in instances such as when a desired point is reached, and negative feedback can be provided in instances such as when a surgeon has moved outside a predetermine parameter. Hands-free 462 navigation involves manipulating the software through gesture control, tool recognition, virtual keypad and the like. Hands-free 462 is done to avoid leaving the sterile field, so it may not be necessary to assign a clinician to operate the computer outside the sterile field.

Data storage 420 can be performed electronically 464 or on paper 466, so information used and developed during the process of surgical navigation can be stored. The stored information can be used for a wide variety of purposes such as monitoring patient recovery and potentially for future patient revisions. The stored data can also be used by institutions performing clinical studies.

Post-operative review and follow-up 422 is typically the final stage in a procedure. As it relates to navigation, the surgeon now has detailed information that he can share with the patient or other clinicians 468.

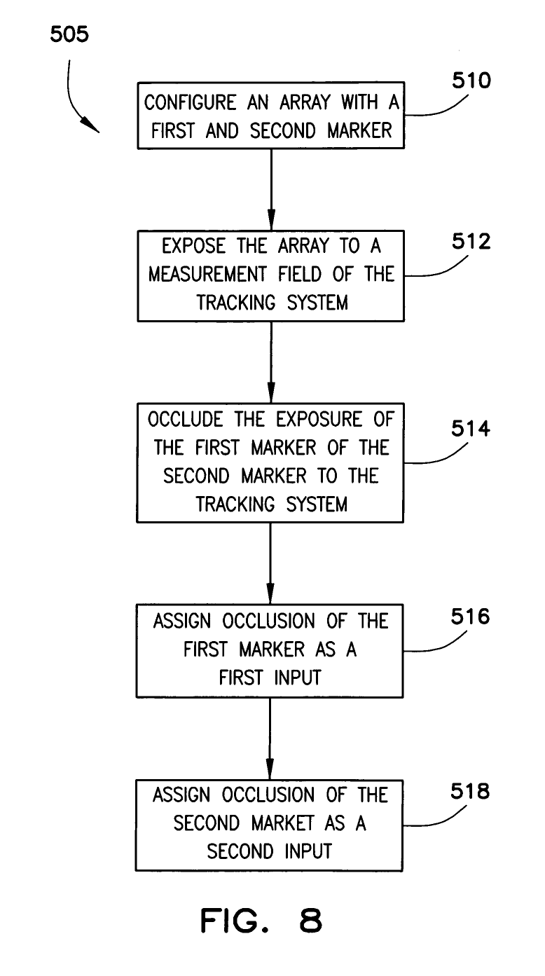

FIG. 8 shows a first flowchart of a selective gesturing embodiment 505. A method for selective gesturing input to a surgical navigation system within a sterile field comprises the following elements. An array is configured with at least a first marker and a second marker 510 but can have additional markers such as a third marker and forth marker. The array can be any array used in surgical navigation such as a probe array, reference array, instrument array, calibration array, and the like. The first marker and second marker distinguishable by a tracking system. The first marker and second marker can be made distinguishable by the tracking system by configuring the array so the markers are arranged in an asymmetric pattern.

The array is exposed to a measurement field of the tracking system 512. The camera is typically positioned so the measurement field extends over a portion or the entire sterile field. The array has a first marker and the second marker that are identified by the tracking system and the position of the array is calculated in an x axis, y axis, and z axis. The orientation of the array can also be calculated by the array's rotation about an x axis, y axis, and z axis. The exposure of the first marker or the second marker is occluded while the markers are exposed to the measurement field within the sterile field 514. The first marker and second marker can be occluded in any sufficient manner such that the tracking system can no longer track the marker. Often a clinician will occlude a marker with her hand.

The occlusion of the first marker is assigned as a first input to a computer system 516, and the second marker is assigned as a second input to the computer system by the tracking system 518. The first input is different than the second input. The first input and the second input can be any inputs relevant to a surgical navigation system such as those inputs shown in the table below, including page forward, page back, tool monitor, help, and the like.

Either the first input or the second input is executed by the computer. The first input and second input can be executed within a single page of an application program to gesturing options. When the first input or second input is executed by the computer system, the computer system will typically provide a visual indication on the computer display of the input being executed.

The following table shows prophetic embodiments of inputs to the computer system. The prophet examples are just of few of the possible inputs, and potentially any touch screen or keyboard input could be configured as a selective gesturing input.

TABLE
Selective Gesturing Examples

| Application | Array | Input |
|---|---|---|
| All | X1 Ref. | Page forward, Page back |
| All but Spine | X2 Ref. | Tool monitor, Help |
| Total Hip | X-Large | Reamer up-size, Reamer down-size |
| Total Hip | Large | Cup up-size, Cup down-size, Save |
| | | cup position, Change implant type |
| Total Hip | Medium | Broach up-size, Broach down-size, |
| | | Save broach position, Change neck |
| | | length |
| Total Hip | Small | Save cut plane, Reset cut plane |
| Total Knee | Large | Save pin location, Reset pin location |
| Uni Knee | Large | Mute sound, Pause burring |
| Uni Knee | Medium | Save posterior cut location, Reset |
| | | posterior cut location |

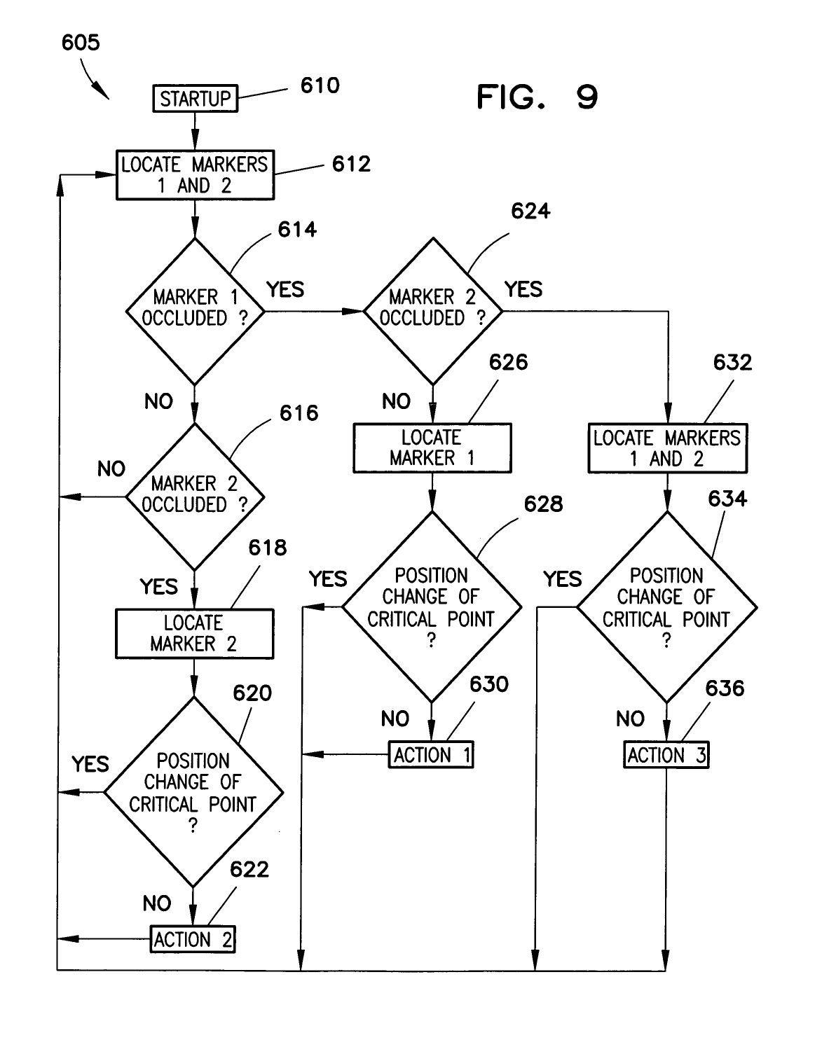

FIG. 9 shows a second flowchart of a selective gesturing embodiment. Some embodiments of selective gesturing can include safeguards to prevent execution of inputs if markers have been unintentionally occluded. In order to determine if a marker has been unintentionally occluded, the system tracks the location of a critical point for each array. A critical point is defined as a point on a device that is known or established with respect to an array. Depending on the device, a critical point for the array may be established during calibration or pre-programmed into the software. The critical point starting position is located, and the critical point finishing position is located within the sterile field. The difference between the critical point starting position and critical point finishing position is calculated to determine if there has been a significant position change. In other words, the distance between the first position and the second position is calculated to determine if the difference exceeds a predetermined value recognized by the computer program. (e.g., 1 mm, 5 mm or the like). If the array has undergone a significant position change during occlusion of the first marker, execution of the first input is prevented. If the array has undergone a significant position change during occlusion of the second marker, execution of the second input is prevented.

More particularly, after the startup (step 610), the tracking system 605 locates markers 1 and 2 (step 612) to determine if either one of the markers is occluded. If marker 1 is not occluded (step 614), then the system 605 checks to see if marker 2 is occluded (step 616). If marker 2 is not occluded, then the system 605 returns to the beginning of the process (step 612). If marker 2 is occluded, then the location of marker 2 is determined (step 618). The system 605 then checks to see if the position of a critical point has changed during the occlusion of maker 2 (step 620) by comparing the current position of the critical point to the previously detected position of the critical point (previous location detected in step 612). If the critical point is located in a different position after being occluded relative to before the occlusion, then the tracking system 605 returns to the beginning of the process (step 612). If the critical point has not moved while occluded, then the tracking system 605 interprets the occlusion as a gesture and proceeds to step 622, which shows performing action 2. Thereafter, the system 605 then returns to the beginning of the process (step 612).

If marker 1 is occluded in step 614, then the system 605 determines if maker 2 is also occluded (step 624). If marker 2 is not occluded, then the tracking system 605 proceeds to step 626 and waits to re-locate marker 1. The system 605 then checks to see if the position of a critical point has changed during the occlusion of maker 1 (step 628). If the critical point changed, then the tracking system 605 returns to the beginning of the process (step 612). If the position of the critical point did not change, then the tracking system 605 interprets the occlusion as a gesture and proceeds to the next step (step 630), which shows performing action 1. Thereafter, system 605 then returns to the beginning of the process (step 612).

If marker 2 is occluded in step 624, then the tracking system 605 proceeds to the next step (step 632) and waits to re-locate markers 1 and 2. The system 605 then checks to see if the position of the critical point changed between before and after occlusion (step 634). If the critical point changed position, then system 605 proceeds back to step 612. If the critical point did not change position, then system 605 proceeds to the next step (step 636), which shows performing action 3. Thereafter, system 605 then returns to step 612.

In exemplary embodiments, the method for selective gesturing input to a surgical navigation system within a sterile field according to the present teachings can be embodied on computer readable storage medium. According to this embodiment, the computer readable storage medium stores instructions that, when executed by a computer, cause the computer to perform selective gesturing in a surgical navigation system. The computer readable storage medium can be any medium suitable for storing instruction that can be executed by a computer such as a compact disc (CD), digital video disc (DVD), flash solid-state memory, hard drive disc, floppy disc, and the like.

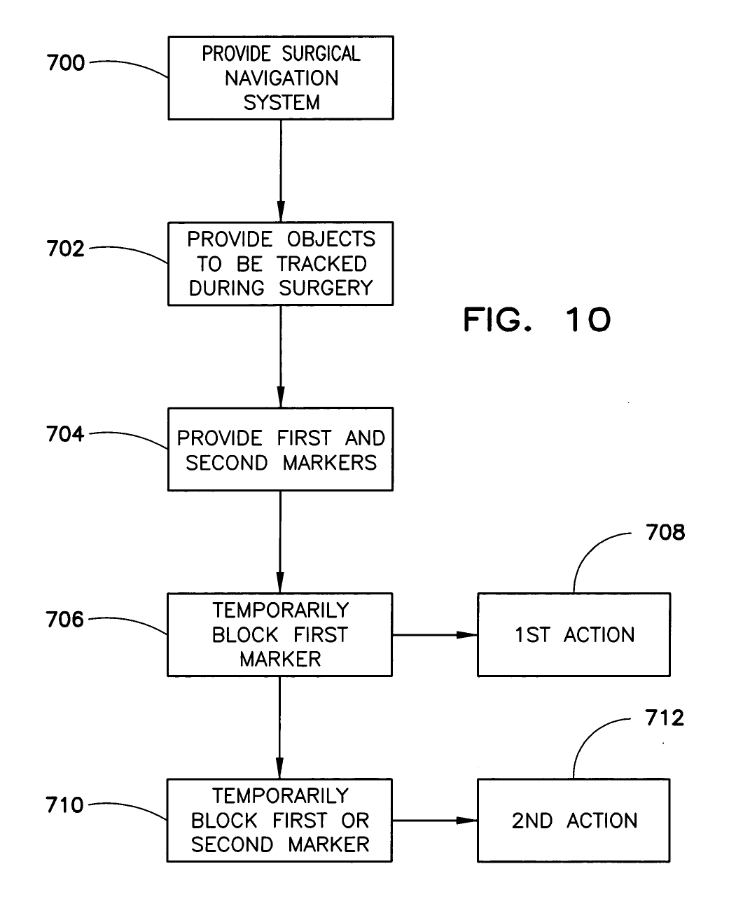

Embodiments incorporating the present teachings enhance image guided surgical procedure by allowing multiple discrete gestures to cause multiple different actions within a single page of surgical protocol. One such embodiment can be appreciated with reference to FIG. 10, in which step 700 illustrates providing a surgical navigation system, such as navigation system 10 of FIG. 1. According to this exemplary embodiment, components for use in this method include the cameras 45 of optical locator 46, which is communicably linked to computer 50 that is programmed with software and includes monitor 32. In step 702, objects that are to be tracked during the surgery are provided, such as probe 22, arrays 24 and 26 and other tools, some of which are shown on tray 34. As noted above, each object has an array attached to it, the array typically having at least three and often four markers. The markers are uniquely identifiable by the software of computer 50. At least two markers are provided for this method, as indicated in step 704.

As shown in step 706, one of the markers is temporarily blocked or occluded. That is, an optical path between a marker and the camera is temporarily blocked, such as by the physician's hand. This causes computer 50 to initiate a first action 708. The first action can be advancing a page on monitor 32, increasing/decreasing the size of an implant or reamer, specifying a distance to be reamed/drilled to name just a few. Alternatively, the first action can be computer 50 prompting the user for a confirmation, thus preventing the possibility of an accidental gesture. As shown in block 710, the method proceeds by either the first or second marker being temporarily blocked, which causes a second action 712 that is different than the first action.

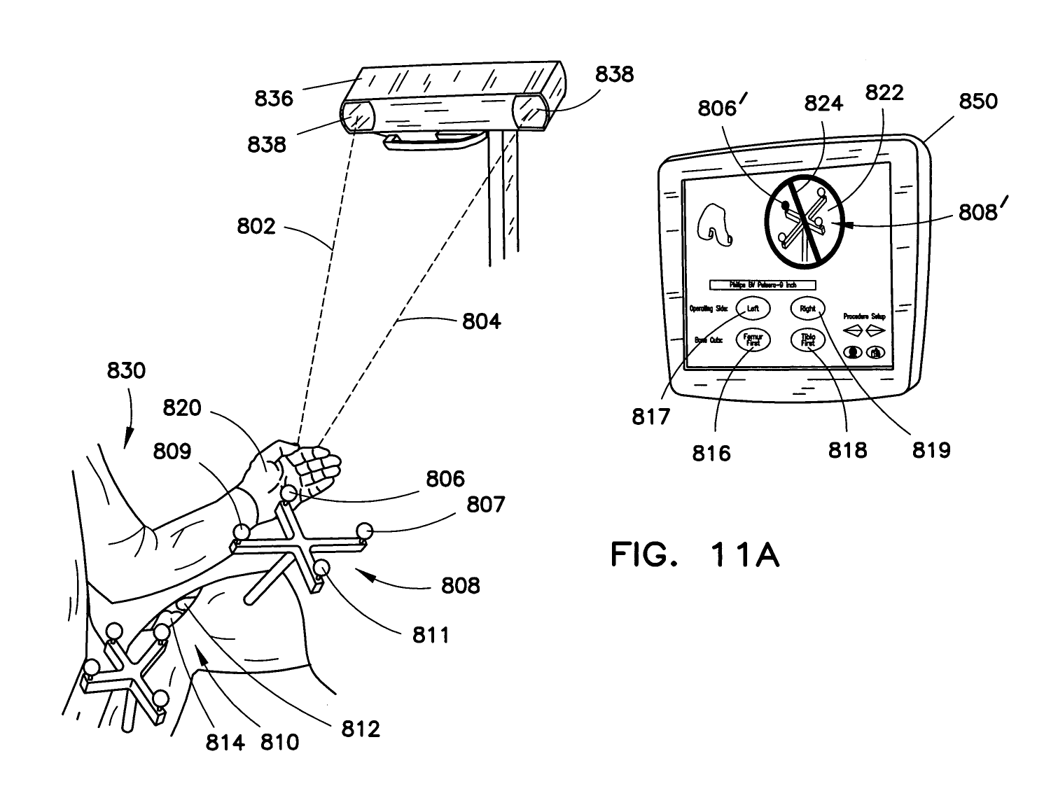

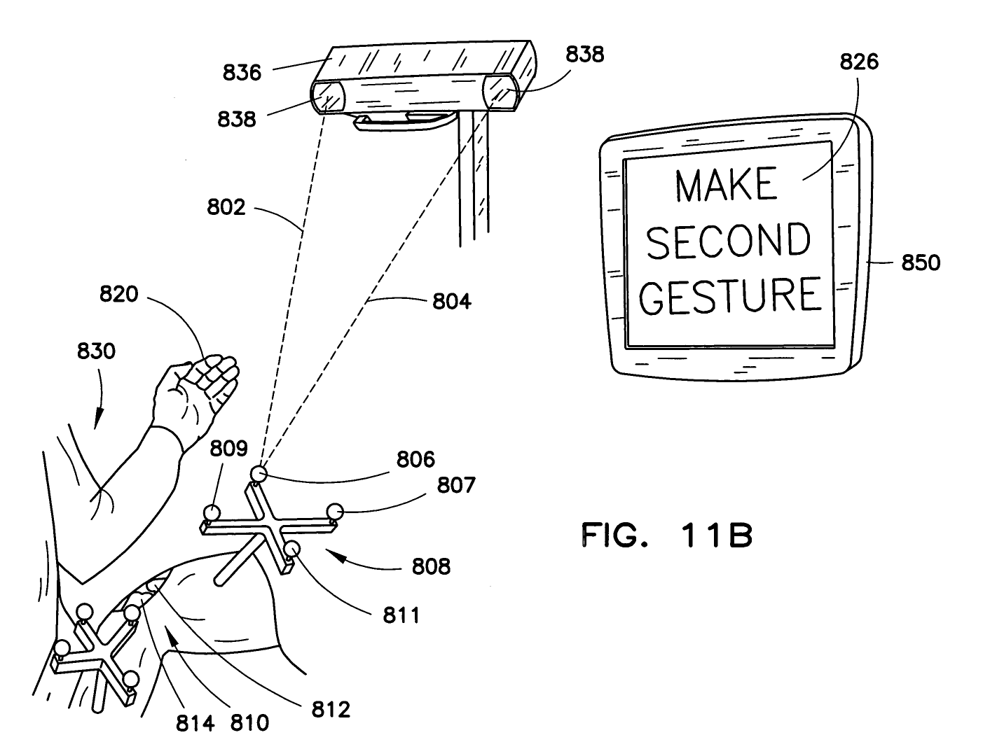

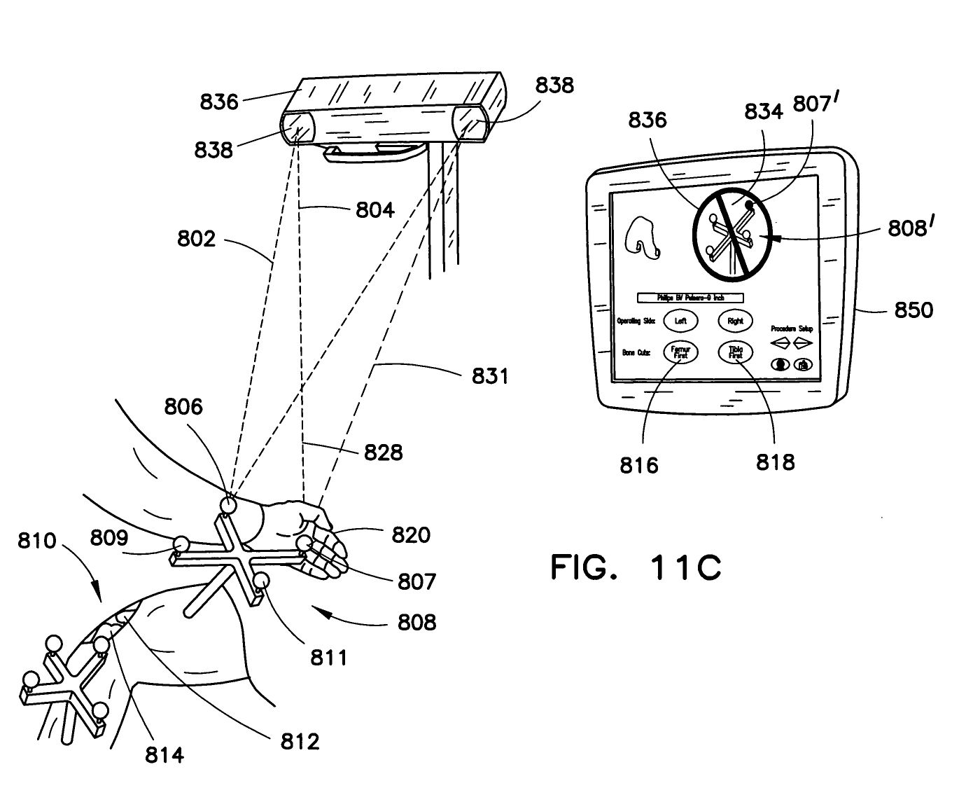

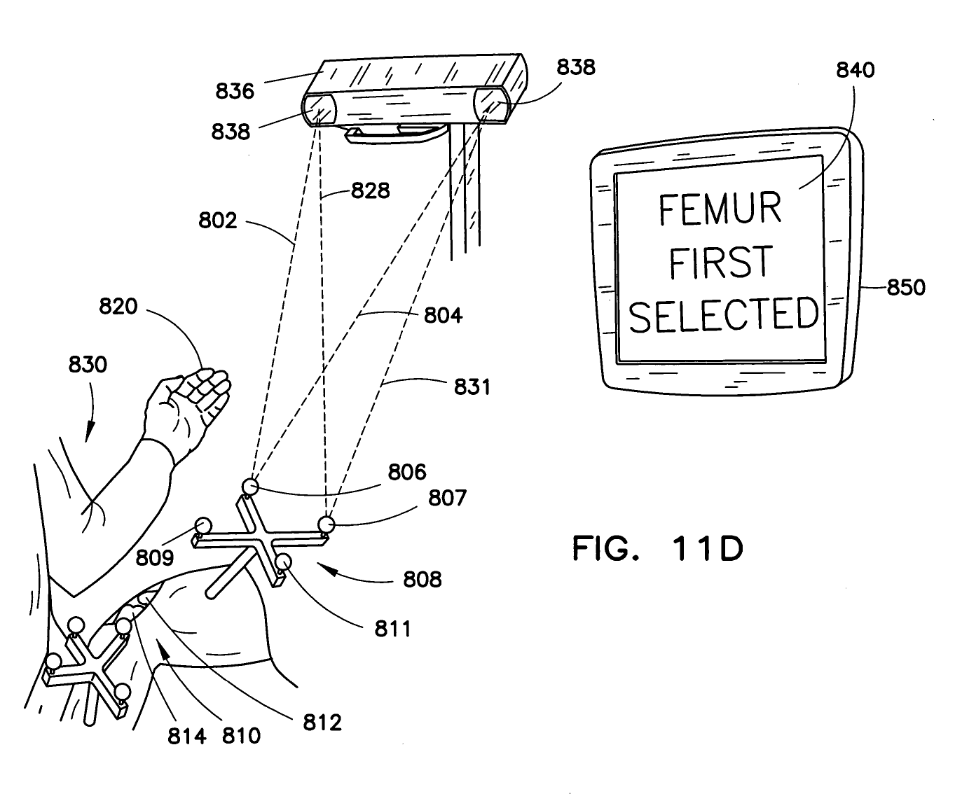

An exemplary example is described with reference to FIGS. 11A-11D. Cameras 838 of optical locator 836 define optical paths 802 and 804 (i.e., define a measurement field of the tracking system) to sphere or marker 806 of array 808, which is exposed to the measurement field of the tracking system. In FIG. 11A, physician 830 is performing a total knee arthroplasty (TKA) procedure on knee 810. Monitor 850 displays a page of surgical protocol in which the physician must choose whether to first cut femur 812 or tibia 814. Conventionally, the physician touches the appropriate icon 816 or 818 on monitor 850. However, such an approach is undesirable because the computer and monitor are located outside the sterile surgical environment.

In the illustrated method, the physician uses his hand 820 to block or occlude the exposure of the optical paths 802 and 804 between marker 806 and cameras 838. (Array 808 also includes spheres 807, 809 and 811 as shown, all of which define optical paths to cameras 838, but which are not shown in FIG. 11A for clarity.) The computer's software acknowledges that sphere 806 has been occluded by assigning the occlusion of the marker as a first input and showing an-image 822 of array 808′ on monitor 850, which depicts occluded marker 806′ as darkened. A circle/slash 824 positioned over array 808′ indicates that array 808 is occluded. After a predetermined amount of time, the monitor will prompt the physician to remove his hand, e.g., by changing the color of circle/slash 824 or changing a color in a status bar.

As shown in FIG. 11B, physician 830 has removed his hand 820 to restore optical paths 802 and 804, which causes monitor 850 to display an image 826 that prompts the physician to make a second gesture. As shown in FIG. 11C, physician's 830 hand 820 is occluding optical paths 828 and 831, i.e., blocking marker 807 from cameras 838. The computer's software acknowledges that sphere 807 has been occluded by assigning the occlusion of the marker as a second input and showing an image 834 of the array 808′ on monitor 850, which depicts the occluded marker 807′ as darkened. The monitor also shows a circle/slash 836 to indicate that array 808′ is occluded. After a predetermined amount of time, the monitor will prompt the physician to remove his hand, as described above.

With reference to FIG. 11D, physician 830 has removed his hand to restore optical paths 828 and 831, which causes monitor 850 to display an image 840 that the femur has been selected for cutting first in the TKA procedure. Thus, in this example of the inventive method, physician 830 has made two gestures, first temporarily blocking sphere 806 and then temporarily blocking sphere 807. The first gesture caused the computer to take a first action, namely, the computer prompted the physician for confirmation. The second gesture caused the computer to take a second action, namely, selecting the femur to be cut first in the TKA procedure.

An example having been described, one of ordinary skill would readily recognize many possibilities for selective gesturing methods in accordance with the present teachings. For example, occluding sphere 807 first, then sphere 806 could cause the tibia instead of the femur to be selected for cutting first. In other embodiments, different gestures cause different actions within the same page of surgical protocol. For example, with reference to FIG. 11A, the software may be programmed such that temporarily occluding sphere 806 a single time (i.e., a single gesture) may cause icon 816 to be activated, thereby selecting the femur for cutting first. The physician may then select icon 819 for the right operating side by temporarily occluding sphere 809 a single time. In this manner, multiple gestures and associated actions are possible within a single screen or page of surgical protocol.

Embodiments incorporating the present teachings are of course not limited to having all markers that are blocked located on a single array or tool. Similarly, in some embodiments, more than one marker may be occluded simultaneously. By the same token, system 10 may be configured such that repeated temporary occlusion of same marker or sphere causes multiple different actions within a single page of surgical protocol. Alternatively, the system may be configures so as to require successively blocking two or more markers to perform a single action. Numerous other variations are possible and would be recognized by one of ordinary skill in the art in view of the teachings above.

Thus, embodiments of the selective gesturing input for a surgical navigation system are disclosed. One skilled in the art will appreciate that the teachings can be practiced with embodiments other than those disclosed. The disclosed embodiments are presented for purposes of illustration and not limitation, and the teachings are only limited by the claims that follow.

What is claimed is:

1. A method for selective gesturing input to a surgical navigation system within a sterile field, comprising:
configuring an array with a first marker and a second marker, wherein the first marker and the second marker are distinguishable by a tracking system;
exposing the array to a measurement field of the tracking system;
occluding the exposure of either the first marker or the second marker to the tracking system within the sterile field; and
assigning the occlusion of the first marker as a first input and assigning the occlusion of the second marker as a second input to the computer system, wherein the first input is different than the second input.

2. The method of claim 1, further comprising:
identifying the array with the tracking system;
calculating a first position of the array before the occlusion;
calculating a second position of the array after the occlusion;
calculating the difference between the first position and the second position; and
preventing execution of the first or second input if the difference exceeds a predetermined value, or executing the first or second input if the difference is less than the predetermined value.

3. The method of claim 1, wherein the first input and second input are executed within a single page of an application program.

4. The method of claim 1, wherein the first input and the second input are selected from the group consisting of page forward, page back, tool monitor, and help.

5. The method of claim 1, wherein the array is a reference array.

6. A computer readable storage medium storing instructions that, when executed by a computer, cause the computer to perform selective gesturing in a surgical navigation system that includes an array having first and second markers and a tracking system, the selective gesturing comprising the following:
identifying the array with the tracking system when the array is exposed to a measurement field of the tracking system; and
recognizing the occlusion of the first marker from the tracking system as a first input and recognizing the occlusion of the second marker from the tracking system as a second input that is different than the first input.

7. The computer readable storage medium of claim 6, wherein the selective gesturing further comprises:
identifying the array with the tracking system;
calculating a first position of the array before the occlusion;
calculating a second position of the array after the occlusion;
calculating the difference between the first position and the second position; and
preventing execution of the first or second input if the difference exceeds a predetermined value, or executing the first or second input if the difference is less than the predetermined value.

8. The computer readable storage medium of claim 6, wherein the first input and second input are executed within a single page of an application program.

9. The computer readable storage medium of claim 6, wherein the first input and the second input are selected from the group consisting of page forward, page back, tool monitor, and help.

10. The computer readable storage medium of claim 6, wherein the array is a reference array.

11. A surgical navigation system, comprising:
a tracking system having a measurement field;
first and second markers that are distinguishable by the tracking system when exposed to the measurement field;
means for recognizing occlusion of the first marker and occlusion of the second marker from the measurement field;
means for causing a first action in response to the occlusion of the first marker; and
means for causing a second action in response to the occlusion of the second marker, wherein the second action is different than the first action.

12. The system of claim 11, wherein the first and second markers are attached to an array.

13. The system of claim 12, further comprising:
means for calculating a first position of the array when exposed to the measurement field;
means for calculating a second position of the array after the exposure of the first or second marker has been temporarily occluded from the measurement field;
means for calculating the difference between the first position and the second position; and
means for preventing execution of the first or second action if the difference exceeds a predetermined value, or executing the first or second action if the difference is less than the predetermined value.

14. The system of claim 11, wherein the first action and second action are executed within a single page of an application program.

15. The system of claim 11, wherein the first action and the second action are selected from the group consisting of page forward, page back, tool monitor, and help.

16. The system of claim 11, wherein the array is a reference array.
